(19) United States
(12) Patent Application Publication
(10) Pub. No.: US 2004/0029164 A1
(43) Pub. Date: Feb. 12, 2004

(54) Protein purification

(76) Inventor: Thomas C. Ransohoff, Lexington, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/455,596

(22) Filed: Jun. 5, 2003

Related U.S. Application Data
(60) Provisional application No. 60/385,964, filed on Jun. 5, 2002.

(51) Int. Cl. C12Q 1/68; C07H 21/04

(52) U.S. Cl. 435/6; 536/25.4

(57) ABSTRACT

The invention feature methods for isolating a recombinant or transgenic polypeptide from egg albumen and a composition containing a transgeenic non-avian polypeptide.

### RELATED APPLICATIONS

This application claims priority to provisional patent application serial No. 60/385,964, filed on Jun. 5, 2002, the entire contents of which are hereby incorporated by reference.

### FIELD OF THE INVENTION

The invention relates to methods of isolating proteins or polypeptides.

### BACKGROUND OF THE INVENTION

A number of commercially important protein molecules, including biosynthetic human insulin, are currently produced on an industrial scale by expression in bacteria, yeast or mammalian cell culture systems followed by recovery and purification. To reduce cost of production and improve scalability for large volume products, approaches for production of proteins in transgenic plant or animal systems have been developed. Avian transgenics systems can be used to produce therapeutic proteins in the white (albumen) fraction of transgenic chicken eggs. However, adequate purification schemes have not been developed.

### SUMMARY OF THE INVENTION

The invention features a method for recovering a polypeptide, e.g., a heterologous polypeptide, from a transgenic avian egg. The method includes the steps of contacting an albumen fraction of the egg with an acid:solvent solution to yield a supernatant from which the polypeptide is recovered. The supernatant is optionally clarified and applied to an adsorptive separation matrix to isolate the polypeptide from the egg.

The polypeptide is acid/solvent-stable. An acid/solvent stable polypeptide is one that it is not substantially degraded or denatured upon brief exposure to solutions containing moderate levels of acid and polar solvent. For example, the activity or structure of the polypeptide is not significantly affected by exposure to at least 10 mM HCl, 10% ethanol for 10 minutes. Less than 50%, preferably less than 40%, preferably less than 25%, and more preferably less than 10% of the biological activity of the polypeptide is lost after exposure to such acid/solvent conditions.

The polypeptide is preferably a non-avian recombinant polypeptide, e.g., a human protein or polypeptide. For example, the human polypeptide is encoded by human DNA and expressed in a non-human transgenic animal, e.g., a bird such as a chicken. Proteins for veterinary use, e.g., dog, cat, horse proteins, produced in hen's eggs are also purified using the described methods. Acid/solvent-stable proteins include insulin or insulin precursors, e.g., a non-avian insulin polypeptide or a non-avian aprotinin polypeptide. The methods are suitable to isolate any proteins which are stable to acid/solvent conditions, e.g., insulin, aprotinin, calcitonin, glucose isomerase, neuromodulin, and anti-neoplastic urinary protein (ANUP). The methods yield a purified transgenic polypeptide that is pharmaceutically-acceptable and suitable for administration to humans.

The acid:solvent solution preferably contains sulfuric acid (H₂SO₄) or hydrochloric acid (HCl); however, other strong acids, e.g., nitric acid and phosphoric acid, are useful. The acid:solvent solution contains an alcohol such as ethanol; however, other polar solvents, e.g., methanol, isopropanol, ethylene glycol and acetone, are also suitable. The method includes a step in which egg albumen, e.g., egg albumen containing a non-avian polypeptide, is diluted into the acid:solvent mixture. A wide range of dilution factors can be used effectively; for example, the ratio of egg albumen to final solution volume is preferably as high as possible, such as 1:4. Alternatively, however, various dilution factors, such as 1:5, 1:7 or 1:10 are also effective. The concentration of the acid in the solution ranges from 0.01N to 1.0N (e.g., 0.01N to 0.2N), and the concentration of the solvent ranges from 1090% (e.g., 18% to 75%). The solution optionally contains a salt, e.g., a sulfate salt, and/or a surfactant, e.g., a non-ionic surfactant.

The albumen fraction is contacted with the acid:solvent solution at a room temperature, e.g., at a temperature of about 20-25 degrees Centrigrade. Alternatively, the contacting step is carried out below room temperature, e.g., at 4 degrees Centrigrade or above room temperature, e.g. at 37 degrees Centigrade. The precipitation temperature may be adjusted to improve yield and/or purity (i.e., removal of egg proteins).

The method optionally includes a step of homogenizing the albumen fraction prior to precipitation. A wide range of shear rates have been demonstrated to be effective at homogenizing egg white. Preferably, the shear rate is sufficient to relatively rapidly and thoroughly homogenize the albumen fraction without damaging (i.e., substantially reducing the biological activity) the non-avian polypeptide, e.g. 5×10⁵ s⁻¹ to 7×10⁶ s⁻¹ (e.g., 6.8×10⁶ s⁻¹). However, if low shear rates are required, the shear rate can be reduced to the level needed to avoid damage to the non-avian polypeptide, e.g., a shear rate as low as approximately 20 s⁻¹.

The method may also contain a centrifugation and/or filtration step after precipitation to remove particulate matter, i.e., clarify, the supernatant prior to further purification. For example, the supernatant is clarified using tangential flow filtration.

After clarification, residual avian polypeptides or proteins such as ovalbumin and conalbumin are removed using one or more purification techniques, such an ion exchange chromatography using an anion exchange resin or a cation exchange resin.

The invention also provides a transgenic non-avian polypeptide composition (e.g., recombinant human insulin purified from a transgenic hen's egg), in which the total avian polypeptides have been collectively reduced to less than 50% of the total amount of avian polypeptides present in unprocessed egg albumen. Per cent reduction is expressed on a w/w basis as compared to the starting egg albumen material. Preferably, the total amount of avian polypeptides is reduced to less than 20%, 10%, 5%, 2% or 1% of the amount of total avian polypeptides on a w/w basis as compared to the starting egg albumen material. For example, the composition contains less than 20 g/L of ovalbumin or less than 5 g/L of conalbumin. The ratio of the concentration of the non-avian transgenic polypeptide to is increased in relation to the concentration compared to the ratio the ratio in unprocessed transgenic egg albumen. For example, the ratio of the non-avian polypeptide to total avian proteins is at least 2-fold greater than the corresponding ratio in an unprocessed transgenic egg albumen. More preferably, the ratio is at least 5-fold, 10-fold, 20-fold, 50-fold, or 100-fold greater compared to the ratio in an unprocessed transgenic egg albumen.

Other features and advantages of the invention will be apparent from the following description of the preferred embodiments thereof, and from the

### BRIEF DESCRIPTION OF THE DRAWINGS

FIG. 1 is a diagram showing steps in a processing method for recovery and purification of heterologous acid/solvent stable protein from egg albumen.

FIG. 2 is a photograph of an electrophoretic gel (SDS-PAGE) showing the results of an acid/solvent-based insulin recovery/purification process from egg albumen.

FIG. 3 is a photograph of an electrophoretic gel (SDS-PAGE) showing insulin cation exchange pools after precipitation using 2 different conditions.

FIG. 4 is a line graph showing the concentration profile of insulin in filtrate during tangential flow filtration.

FIG. 5 is a photograph of an electrophoretic gel (SDS-PAGE) showing the results of acid/solvent-based precipitations of insulin-containing egg albumen using different precipitation conditions.

FIG. 6 is a photograph of an electrophoretic gel (SDS-PAGE) showing effective recovery of aprotinin after acid-solvent precipitation-based aprotinin recovery from egg albumen.

### DETAILED DESCRIPTION

Recombinant non-avian polypeptides expressed and manufactured in avian tissues, e.g., hen's eggs, are purified to remove avian impurities. Production of recombinant polypeptides using avian transgenics has several advantages compared to other methods of producing recombinant proteins, e.g., reduced capital and operating costs, speed of founder development and production flock scale-up, containment of the production species. The methods described herein allow cost-effective recovery and purification of heterologous proteins from egg albumen on a large scale. Additionally, the techniques meet a variety of processing requirements to enable production of human therapeutic compounds on the multi-kg up to ton scale.

To meet the stringent purity requirements for human therapeutic applications, including clearance of host proteins and pathogens, selective adsorption, such as chromatography, is used. The high level of proteins in egg albumen (approximately 100 g/L) places a much higher burden on selectivity of the initial purification steps compared to other therapeutic protein feed streams. For example, mammalian cell culture supernatants from serum free media can have a protein background of <1 g/L and goat's milk has a protein background of approximately 30 g/L.

Drawbacks of earlier protein purification methods include fouling or plugging of the absorptive separation media due to irreversible non-specific binding during application of the sample to be processed. Another drawback of previous methods is the inherently lower efficiency of chromatography operations when purifying material from lower purity feed streams. In order to effectively operate chromatography at process scale, the load solution is homogeneous, clarified, and treated to render the solution non-fouling and reduce the avian protein background. The adsorptive media can be used for a plurality of cycles of loading, washing, product elution and regeneration.

The invention provides cost-effective way to prepare a process-suitable load for a chromatography column from egg albumen to enable production of high purity proteins from an avian transgenic source. Additionally, the invention provides a means to provide a high level of selectivity for enriching the heterologous protein over the egg albumen proteins during the recovery and initial purification steps. The methods described herein are broadly-applicable methods to achieve acceptable levels of purity of a wide range of proteins/polypeptides from egg albumen.

The methods are suitable for broad classes of proteins. For example, the invention is useful for recovery and purification of proteins that are soluble and relatively stable in acid/solvent conditions. Examples of such proteins include insulin, calcitonin, aprotinin (bovine pancreatic trypsin inhibitor). Compared to other proteins, this class of proteins withstands aggressive solution conditions (e.g., acidic or solvent conditions) for short periods of time during processing or analysis without significant denaturation or loss of activity.

Activity of isolated proteins or polypeptides is determined using methods known in the art. For example, insulin is assayed for bioactivity using a free fat cell assay that is based on incorporation of tritiated glucose into lipids during incubation with free murine fat cells (e.g., Moody et al., 1974, Horm Metab Res. 6(1): 12-16; Brange et al., 1990, Diabetes Care; 13(9):923-954). Insulin polypeptides expressed in avian tissues are typically inactive insulin precursor molecules. Thus, insulin bioactivity is measured after cleavage and transpeptidation of the insulin precursor (e.g., proinsulin) that is expressed in avian egg albumen. Insulin activity is measured after processing the transgenic gene product recovered from albumen. The activity of aprotinin or other enzyme inhibitors is measured by measuring inhibtion of trypsin enzymatic activity, e.g., using the method described by Kassell et al., 1970, Methods Enzymol.; 19: 844-852 or Ferrer et al., 1992, Int. J. Peptide Protein Res.; 40: 194-207. This standard assay is based on inhibition of tryptic hydrolosis of N-a-benzoyl-DL-arginine-p-nitroanilide (BAPA), which results in an increase of absorbance at 405 nm. Assays to measure the activity of other target proteins are well known in the art.

Recovery and Purification of Acid-Stable Polypeptides

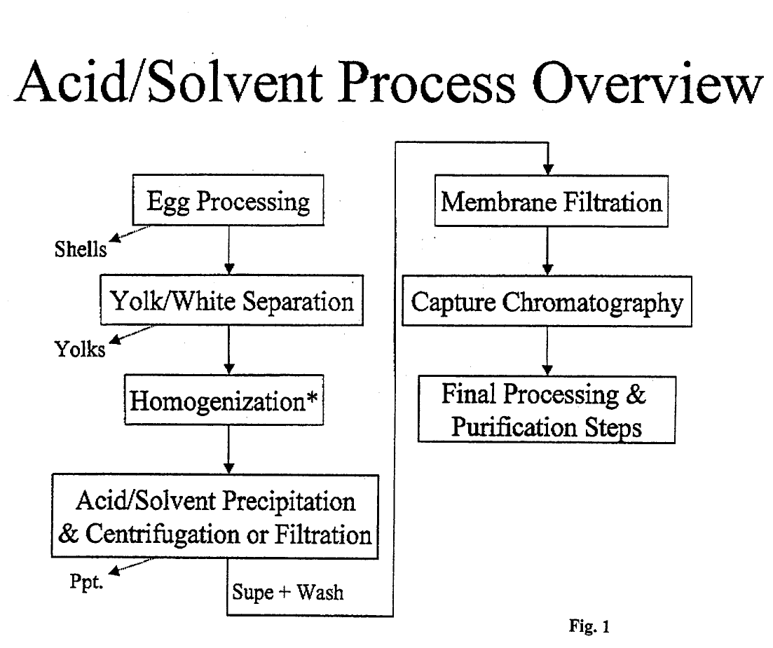

The protein purification and processing method allows the recovery and initial purification of acid-stable proteins that are soluble and stable in moderate to high amounts of water-miscible solvents, (e.g., ethanol) from egg albumen. The method includes optionally mixing or homogenizing the egg albumen early in the processing scheme (e.g., prior to precipitation) to reduce viscosity and improve fluid handling. A significant percentage of the background egg proteins is removed by a precipitation step. The egg albumen is diluted into an acid:solvent solution, and the heterologous protein (i.e., the recombinant or transgenic target protein or polypeptide) is retained in solution. The precipitated proteins (e.g., contaminating avian proteins) are separated from the solution using known methods, e.g., centrifugation and/or filtration, to yield a clarified supernatant. The clarified supernatant is suitable for application onto an adsorptive separation matrix, such as chromatographic resin or expanded bed media, without excessive fouling. FIG. 1 shows an overview of the processing method.

Homogenization of Egg Albumen

Transgenic hens' egg albumen is optionally homogenized prior to precipitation. The egg albumen fraction of a chicken egg was separated from egg yolk using a standard egg separator, similar to an egg separating cup on a commercially available large-scale separator. After separation, the recovered albumen fraction is heterogeneous, containing some solids, a “thick” fraction, and a “thin” fraction. The thick fraction is relatively viscous, having a viscosity of approximately 20 cp at a shear rate of 24 s⁻¹. By reducing the viscosity of the thick fraction and making the solution homogeneous, the ability to manage the egg albumen during further processing was improved.

The data described herein demonstrates the ability to reduce viscosity and provide a homogeneous solution using mechanical mixing at surprisingly low shear rates. Initial work on homogenization was conducted using a microfluidizer (Microfluidics, Inc., Newton, Mass.). Egg albumen was processed on a Microfluidizer M-110Y using 1-3 passes in processing conditions ranging from 2 kpsi to 20 kpsi. All conditions provided a homogeneous effluent with a viscosity of approximately 1 cp. Material processed at 10 kpsi was filtered more readily through a Cuno Zeta Plus 05SP depth filter than material processed at 2 kpsi; however, both conditions provided a homogeneous solution with a viscosity close to water that would be suitable for further processing. Shear rates for these processes range from 5 to 7×10⁶ s⁻¹, e.g., 5.1 to 6.8×10⁶ s⁻¹. Although this method is highly effective at homogenization and viscosity reduction of the egg albumen, some proteins/polypeptides may be rendered denatured or otherwise compromised (e.g., lower specific activity) with the high shear rates used.

Alternative homogenization methods were also evaluated. Surprisingly, a homogeneous solution with viscosity close to water was also achieved using a relatively low shear rate, e.g., using a lab stir-plate mixer operating for at a shear rate of approximately 23 s⁻¹ for about 60 minutes. Low shear rate homogenization is desirable for heterologous proteins that are sensitive to denaturation at high shear rates. Finally, a rotor-stator type homogenizer was tested and found to provide effective and rapid homogenization at intermediate shear rates. Egg albumen was processed using an Ultra-Turrax T8 homogenizer with an S8N-8G dispersing element (IKA-Werke, Staufen, Germany) for approximately 1 minute to provide a homogeneous solution with reduced viscosity. The data indicated that viscosity reduction and homogenization was achieved using any of a number of mechanical mixing techniques over a wide range of shear rates, including those with low shear rates such as static mixing.

Precipitation, Centrifugation/Filtration, and Chromatographic Purification of Human Insulin from Hen's Egg Albumen

While homogenization reduces viscosity and provides a homogeneous liquid, the resulting egg albumen solution, even after dilution with up to 10 volumes of a suitable buffer, such as PBS, can plug membrane filters (dead-end and tangential flow), chromatography columns, and even expanded bed adsorption devices. Precipitation techniques were found to improve filterability.

The data showed that that precipitation of a significant fraction of the egg albumen proteins was achieved by diluting the processed egg albumen into an acid:solvent solution. Because the desired heterologous protein remains predominantly soluble in the supernatant, a significant purification/enrichment of the desired protein was achieved in this step.

The initial recovery and purification steps enable the first chromatography column to capture the heterologous protein (or, alternatively, the avian protein impurities) with minimal adjustment of solution conditions. Once loaded onto the first chromatography column, standard processing methods and techniques are used to purify the product to ensure that the product meets purity specifications. The methods for primary recovery and capture purification yield a composition containing a recombinant or transgenic polypeptide, the level of purity of which is at least 20-fold greater than the level of purity of the starting material (e.g., transgenic egg albumen). Greater than a 50-fold or 100-fold increases in purity are achieved using the described methods. For example, 2-3 mg/ml of a transgenic human polypeptide (e.g., human insulin) is initially present in a transgenic hen's egg (approximately 2.5% purity on a total protein w/w basis). Following processing through precipitation and capture purification on a cation exchange column, the % purity is 50% or more.

### EXAMPLE 1

### Purification of Human Insulin from Egg Albumen

Insulin was used as a model heterologous acid/solvent stable protein. To prepare a spiked egg albumen solution, a stock solution of recombinant human insulin (Insulin [Sigma P/N I-2018] at 20 mg/ml in 0.01N HCl) was spiked into egg albumen at a dilution of 1:8.5. The resulting concentration of insulin in egg albumen was approximately 2.3 mg/ml.

The spiked egg albumen was diluted 1:10 into the precipitation solution, 0.2N HCl, 75% ethanol. The precipitation was allowed to occur for approximately 15 minutes at room temperature with gentle shaking. On completion of the precipitation, the samples were centrifuged at approximately 2000×g for 10 minutes. The supernatant was recovered and the pellet was washed with 10 volumes of 0.1M HOAc, pH 4.0 for 50 minutes. The material was centrifuged again at approximately 2000×g for 10 minutes, and the supernatant was recovered.

The pooled supernatant was filtered using a dead-end 0.45 micron membrane filter and adjusted to pH 4.0 using NaOH. The material was then be loaded directly to a cation-exchange chromatography column as the first step in the final purification process. The cation exchange step is optionally followed by an anion exchange chromatography step and a polymeric reversed phase step to provide a 3-step chromatography process, which recovered insulin to very high purity (>98% by HPLC).

The level of purity was determined as follows. In one example, a sample of an initial egg albumen solution of 33 ml spiked with 75 mg of recombinant human insulin was purified by precipitation and cation exchange chromatography as a capture purification step.

The initial insulin concentration was calculated from the amount spiked as approximately 2.3 mg/ml; the initial total protein concentration was estimated based on a value of approximately 102 mg/mL protein in egg albumen and the spike amount to be approximately 91 mg/ml. Therefore, the initial insulin purity on a w/w basis was approximately 2.5%.

In the cation exchange pool recovered after precipitation and cation exchange purification of the starting spiked solution, the total protein concentration was determined by BCA assay to be approximately 0.475 mg/ml; whereas the insulin concentration was determined by HPLC to be approximately 0.244 mg/ml. Therefore, the insulin purity in the recovered solution was approximately 51%, or an improvement in purity of over 20-fold from the starting solution.)

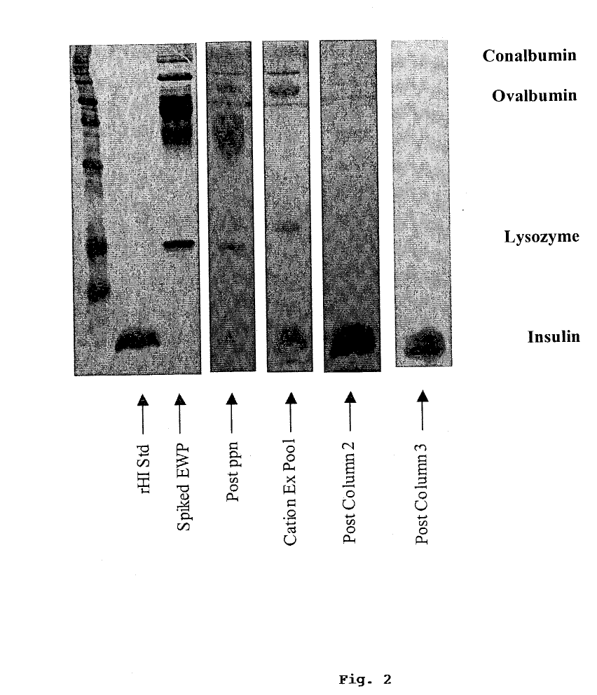

The method effectively removes contaminating avian egg proteins. Ovalbumin and conalbumin are the two most abundant egg proteins representing approximately 66% of the total egg albumen protein. FIG. 2 is a photograph of a silver-stained SDS-PAGE gel showing the level of recovery during the precipitation process and subsequent chromatography steps. As shown in the gel, the precipitation process described herein removes a significant percentage of the egg proteins, including most of the ovalbumin and conalbumin, prior to the first chromatography step. This fractionation step both improves filterability and provides a significant purification/enrichment from the major egg protein contaminants, enabling development of an efficient chromatography process for final purification.

A reversed-phase HPLC assay (YMC Butyl 5μ, 300 A) was used to quantify insulin recovery from the process as shown in Table 1. The overall yield of the process is approximately 40%. The yield is improved by standard optimization techniques.

TABLE 1

| Sample | N | Ave. Recovery |
|---|---|---|
| Spiked Egg Albumen | N/A | N/A |
| Cation-Exchange Load (post precipitation) | 1 | 63% |
| Cation-Exchange Pool | 4 | 80% +/− 20% |
| Anion-Exchange Pool | 2 | 80% +/− 8% |
| Reversed Phase Pool | 2 | 96% +/− 5% |

### EXAMPLE 2

### Acid/Solvent Conditions for Purification of Non-Avian Proteins from Hen's Egg Albumen

Precipitation conditions are adjusted over a relatively wide range to provide a tailored result. Among the parameters that can be influenced by the precipitation conditions are the degree of purification achieved, the yield of heterologous protein, the cost of the precipitation process, and the filterability of the resulting supernatant.

A recombinant human insulin in hen's egg albumen (e.g., 2 g/L) was subjected to a variety of precipitation conditions (Table 2). The solution was homogenized using low shear mechanical mixing. The solution was then diluted with a number of different acid:solvent precipitation solutions at different dilution ratios and different precipitation incubation temperatures. The supernatants from these precipitations were centrifuged as described above, filtered through a 0.45μ filter, and analyzed for insulin concentration using the HPLC assay described above. The insulin yields and filterability resulting from the various precipitation methods are summarized in Table 2.

TABLE 2

| | | Filterability |
|---|---|---|
| Precipitation Conditions | Overall Yield | (L/m²) |
| 0.2N HCl, 75% EtOH, 1:10, RT | 27% | 38 |
| 0.2N HCl, 30% EtOH, 1:10, RT | 29% | 20 |
| 0.05N HCl, 75% EtOH, 1:10, RT | 24% | 25 |
| 0.05N HCl, 30% EtOH, 1:10, RT | 47% | 13 |
| 0.05N HCl, 18% EtOH, 1:10, RT | 70% | 13 |
| 0.05N HCl, 18% EtOH, 1:10, 4C | 74% | 10 |
| 0.05N HCl, 18% EtOH, 1:5, RT | 42% | 10 |
| 0.2N HCl, 18% EtOH, 1:5, RT | 57% | 20 |

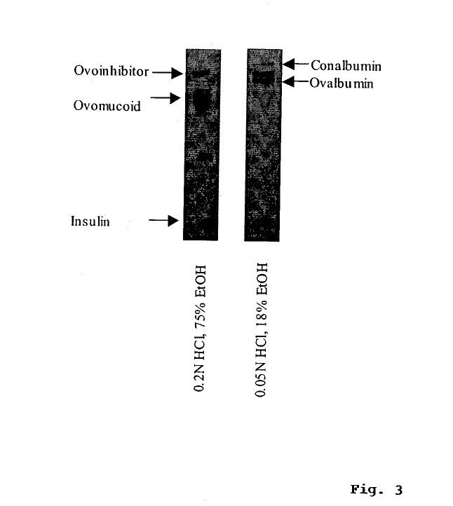

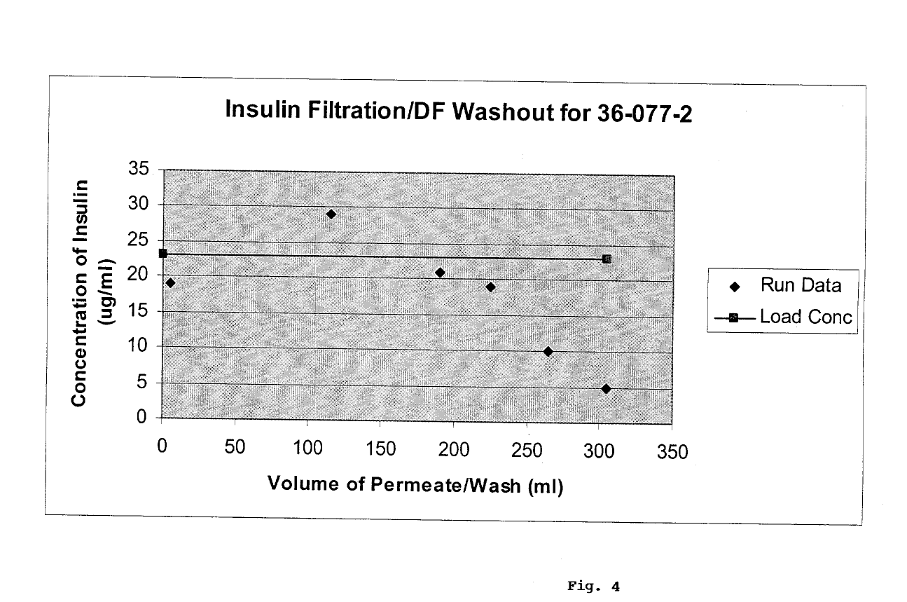

To evaluate the impurity profile resulting from the various precipitation methods, solutions from the most aggressive (0.2N HCl, 75% EtOH) and least aggressive (0.05N HCl, 18% EtOH) were processed through tangential flow filtration and the cation exchange step (FIGS. 3-4).

While both materials were suitable for filtration by tangential flow microfiltration and loading onto the ion exchange chromatography column, there was a significant difference in the impurity profile in the resulting cation exchange pool (FIG. 3). Specifically, more ovalbumin and conalbumin is carried through to the cation exchange load and pool with the less aggressive precipitation conditions. A range of acid/solvent precipitation conditions is the basis of an effective recovery and purification process, and conditions may be optimized for any particular target protein or polypeptide. Fine tuning of precipitation conditions is carried out using standard methods. For example, for human insulin, less aggressive precipitation conditions resulted in higher yields but lower filterability and initial purification from egg proteins.

### EXAMPLE 3

### Tangential Flow Filtration for Clarification of Post-Precipitation Supernatants

While the acid:solvent precipitation method provides a filterable supernatant after centrifugation, “dead-end” membrane filtration of the supernatant may result in high processing costs and frequent filter changes. Factors, which affect filterability include stringency of homogenization and precipitation conditions used; less stringent homogenization and/or precipitation conditions will generally result in supernatants that are more challenging to filter. Membrane filter life is improved substantially by pre-filtering with a depth filter; however, even after some optimization, these filters may plug at high throughputs. Alternative and/or additional filtration approaches were evaluated to reduce processing costs.

Tangential flow filtration, using processing conditions designed to enable passage of the heterologous protein through the membrane, is a cost-effective means of clarifying the post-precipitation supernatant prior to the first chromatography column. This method also lends itself to scaling up to commercially-relevant production levels, e.g., kg to ton scale.

To perform the separation, a tangential flow filtration device was used. The membrane used was a 0.1 micron hollow fiber microfilter (A/G Technology, CFP-1-E-MM01A, Newton, Mass.). The starting material was a supernatant from a low shear homogenization (approximately 25 s⁻¹) followed by precipitation using 0.2N HCl, 75% EtOH and a 1:10 dilution as the precipitation conditions. The material was centrifuged, and the resulting supernatant was recovered and diluted 1:2 with 50 mM HOAc, pH 4.0.

The diluted supernatant was processed by tangential flow filtration using a hollow fiber microfilter under conditions that promote passage of the product through the membrane and provide reasonable filtration rates. A recirculation rate of approximately 4100 l/m²/h, and a trans-membrane pressure of 6-10 psig was used. Flux rates obtained using these conditions averaged approximately 80 l/m²/h during the clarification process.

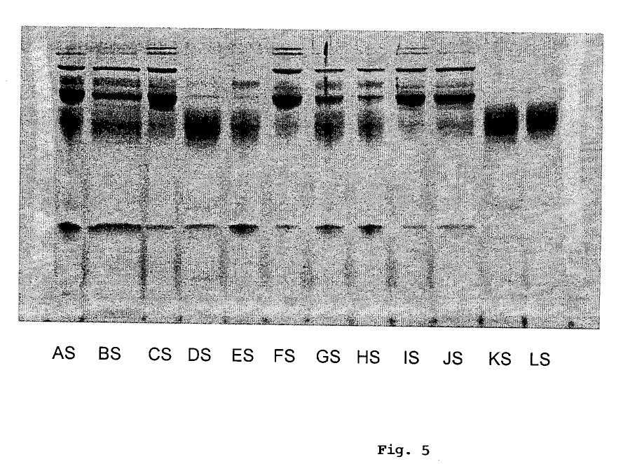

After over 80% of the material had been clarified, the remaining retentate was washed with three volumes of 50 mM HOAc, pH 4.0 to wash insulin remaining in the retentate through the membrane. Throughout the process, samples of the filtrate were taken for evaluation of insulin concentration by HPLC. FIG. 5 shows a graph of insulin concentration as a function of filtrate volume. This graph shows a stable clarification of insulin during the concentration phase with the expected washout of insulin during the wash phase. Overall recovery of insulin through the filtration process, as determined by HPLC, was approximately 100%.

This type of filtration process works with a range of tangential flow ultrafiltration or microfiltration membranes. The most cost-effective combination of membrane material and operating conditions for each specific application are defined and optimized using standard methods. Since tangential flow filtration membranes can be cleaned and re-used for many cycles, this type of filtration process is significantly more cost-effective when used in conjunction with precipitation and centrifugation steps rather than the depth/dead-end filtration processes previously reported.

### EXAMPLE 4

### Purification of an Acid/Solvent Stable Protein:Insulin

A variety of acids were evaluated to identify acids suitable for precipitating egg albumen proteins while retaining human insulin in the supernatant fraction. For example, hydrochloric acid, sulfuric acid, nitric acid and phosphoric acid were investigated at concentrations ranging from 50 to 100 mM. Ethanol was used at concentrations from between 0%, 25%, and 50%. Additionally, the addition of other salts (MgSO₄ and (NH₄)₂SO₄) was evaluated.

All samples used a homogenized (low shear rate method) egg albumen containing 1 g/L recombinant insulin as the starting material. This material was diluted with the acid:solvent mixture 1:10 and precipitation was allowed to occur for approximately 20 minutes at room temperature. The samples were centrifuged for 5 minutes at 13,200 rpm in an Eppendorf centrifuge. The supernatants were evaluated using BCA analysis for total protein content, Sypro Ruby-stained 18% SDS-PAGE, and HPLC for insulin recovery.

Table 3 shows the calculated reduction of total protein and insulin recovery based on the BCA and HPLC results. The increase in insulin purity was also calculated and tabulated.

TABLE 3

| | | Total Protein | Insulin | Increase in |
|---|---|---|---|---|
| Sample | Precipitation Conditions | Reduction | Recovery | Purity |
| AS | 100 mM HCl, 50% | 87% | 74% | 5.7x |
| | ethanol | | | |
| BS | 100 mM HCl, 25% | 76% | 53% | 2.2x |
| | ethanol | | | |
| CS | 100 mM HCl, 0% | NM (<0%) | ND | <1x |
| | ethanol | | | |
| DS | 50 mM H₂SO₄, 50% | 95% | 56% | 11x |
| | ethanol | | | |
| ES | 50 mM H₂SO₄, 25% | 87% | 55% | 4.2x |
| | ethanol | | | |
| FS | 50 mM H₂SO₄, 0% | NM (<0%) | ND | <1x |
| | ethanol | | | |
| GS | 100 mM HNO₃, 50% | 88% | 61% | 5.1x |
| | ethanol | | | |
| HS | 100 mM HNO₃, 25% | 84% | 61% | 3.8x |
| | ethanol | | | |
| IS | 100 mM HNO₃, 0% | NM (<0%) | ND | <1x |
| | ethanol | | | |
| JS | 50 mM H₃PO₄, 50% | 60% | 63% | 1.6x |
| | ethanol | | | |
| KS | 50 mM H₂SO₄, 50 mM | 94% | 77% | 13x |
| | MgSO₄, 50% ethanol | | | |
| LS | 50 mM H₂SO₄, 50 mM | 93% | 68% | 9.8x |
| | (NH₄)₂SO₄, 50% ethanol | | | |

Supernatants were analyzed by silver-stained SDS-PAGE. While all of the acids tested were effective for fractionation, the SDS-PAGE, BCA, and HPLC results indicate that sulfuric acid was preferred. That data indicated that at least 1% of an alcohol, e.g., ethanol, was required to achieve a desired measure of purification, and the higher concentration of ethanol (50%) provided improved purification for each acid tested. Addition of 50 mM MgSO₄ or (NH₄)₂SO₄ improved insulin recovery as compared to using the corresponding acid, e.g., H₂SO₄, alone. These data indicated that purification is increased by adding a salt.

### EXAMPLE 5

### Dilution Ratios of Acid/Solvent and Precipitation Cofactors

A range of different dilution ratios of acid/solvents in the presence/absence of different additives (e.g., salts or surfactants) were evaluated to identify preferred acid:solvent compositions (e.g., 100 mM H₂SO₄, 60% ethanol) to precipitate egg albumen proteins while retaining human insulin in the supernatant fraction. For example, the ratio of insulin-containing egg albumen:final solution volume was varied between 1:4, 1:7 and 1:10. The addition of other salts (50 mM and 100 mM MgSO₄ and 50 mM (NH₄)₂SO₄) and of 0.05% Tween-20 was evaluated.

All samples used a homogenized (intermediate shear rate method, using a Ultra-Turrax rotor-stator homogenizer) egg albumen spiked with 1 g/L recombinant insulin as the starting material. This mixture was diluted with the acid:solvent mixture at the dilution ratio indicated in Table 4 and precipitation was allowed to occur for approximately 20-25 minutes at room temperature. The samples were centrifuged for 5 minutes at 13,200 rpm in an Eppendorf centrifuge. The supernatants were evaluated using HPLC analysis for insulin recovery and as an indicator for purification achieved. The purity indicating factor was calculated by dividing the insulin peak area with the total peak area of two known impurity peaks. A higher purity indicating factor would correlate to higher purity in the recovered supernatant

Table 4 below shows the calculated insulin recovery and purity indicating factor based on the HPLC results.

TABLE 4

| | Dilution Factor | Insulin | Purity |
|---|---|---|---|
| | (Egg Albumen | Re- | Indicating |
| Precipitation Conditions | vol:Final Soln vol) | covery | Factor |
| 100 mM H₂SO₄, 60% ethanol | 1:4 | 92% | 4.0 |
| 100 mM H₂SO₄, 60% ethanol | 1:7 | 89% | 0.55 |
| 100 mM H₂SO₄, 60% ethanol | 1:10 | 77% | 0.45 |
| 100 mM H₂SO₄, 60% ethanol, | 1:7 | 85% | 1.2 |
| 50 mM MgSO4 | | | |
| 100 mM H₂SO₄, 60% ethanol, | 1:7 | 100% | 1.7 |
| 100 mM MgSO4 | | | |
| 100 mM H₂SO₄, 60% ethanol, | 1:7 | 73% | 1.1 |
| 50 mM (NH₄)₂SO₄ | | | |
| 100 mM H₂SO₄, 60% ethanol, | 1:7 | 78% | 0.85 |
| 0.05% Tween-20 | | | |

The data showed that, while all conditions provide good yield and significant purification, a lower dilution factor is preferred as it improves insulin yield and purity and is likely to be less costly. Addition of the salts tested and of a non-ionic surfactant provides purity improvements at similar yields. Specific ratios of cofactors is optimized for specific target molecules using standard methods. Salts promote further precipitation of egg proteins, whereas non-ionic surfactants stabilize non-avian target polypeptides without significantly affecting the precipitation of egg proteins.

### EXAMPLE 6

### Purification of an Acid/Solvent-Stable Protein: Aprotinin

Aprotinin (bovine pancreatic trypsin inhibitor) is a serine proteinase inhibitor that is able to preserve adhesive glycoproteins in the platelet membranes and has been approved for a number of clinical indications. For example, aprotinin is used as an antifibrinolytic and to reduce transfusion requirements during surgical procedures, e.g., cardiac surgery. Transgenically-produced aprotinin or fragments thereof are produced trangenically in hen's egg albumen.

To demonstrate the ability of the described methods to be used for other acid/solvent stable proteins, a second acid/solvent-stable protein, aprotinin, was purified. Aprotinin (bovine pancreatic trypsin inhibitor) was obtained from Sigma and dissolved in 0.1M acetic acid to produce a 20 mg/ml stock solution. To produce a spiked egg albumen mixture containing aprotinin at 1 mg/ml, the stock solution was diluted 1:20 with egg albumen. The mixture was homogenized using an intermediate shear rate as described above. The egg albumen mixture was diluted 1:4 into an acid solvent solution, to achieve a precipitation condition of 100 mM H₂SO₄, 60% ethanol. A control egg albumen mixture (containing no aprotinin) was precipitated using the same procedure. The precipitation of both samples was allowed to occur for approximately 30 minutes at room temperature. The samples were centrifuged for 5 minutes at 13,200 rpm in an Eppendorf centrifuge.

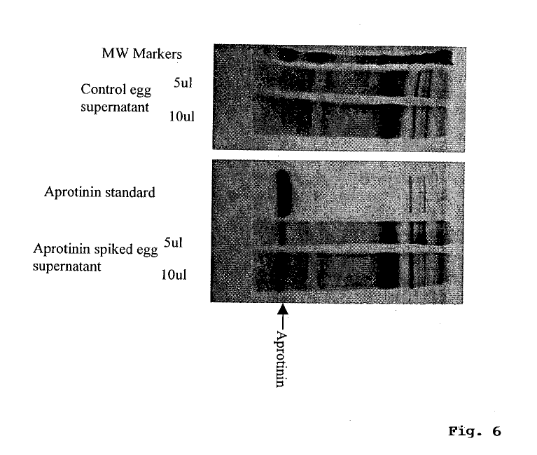

The supernatants were evaluated using SDS-PAGE and HPLC analysis to evaluate recovery of aprotinin. HPLC analysis indicated that 88% of aprotinin was recovered in the supernatant. SDS-PAGE was run on samples that had been concentrated with a vacuum drying system and resuspended in 0.5M Tris, pH 8. The silver-stained 18% SDS-PAGE gel in FIG. 6 indicated successful recovery of the aprotinin from egg albumen in the supernatant. and a similar impurity profile (residual egg protein bands) compared to the control egg albumen sample.

Other embodiments are within the following claims.

What is claimed is:

1. A method for recovering a polypeptide from a transgenic avian egg, comprising contacting an albumen fraction of said egg with an acid:solvent solution to yield a precipitate and a supernatant and recovering said polypeptide from said supernatant.

2. The method of claim 1, wherein said polypeptide is acid:solvent stable.

3. The method of claim 1, wherein said polypeptide is non-avian.

4. The method of claim 1, wherein said polypeptide is a non-avian insulin or insulin precursor polypeptide.

5. The method of claim 1, wherein said polypeptide is a non-avian aprotinin polypeptide.

6. The method of claim 1, wherein said acid: solvent solution comprises H₂SO₄.

7. The method of claim 1, wherein said solvent is polar.

8. The method of claim 1, wherein said acid:solvent solution comprises ethanol.

9. The method of claim 1, wherein said albumen fraction is diluted into said acid:solvent solution to to yield a ratio of 1:4 after dilution.

10. The method of claim 1, wherein said albumen fraction is diluted into said acid:solvent solution to to yield a ratio of 1:4 after dilution.

11. The method of claim 1, wherein said acid:solvent solution comprises a concentration of said acid in the range of 0.01N to 0.1N.

12. The method of claim 1, wherein said acid:solvent solution comprises a concentration of said solvent in the range of 10% to 90% of said solution.

13. The method of claim 1, wherein said contacting is carried out at room temperature.

14. The method of claim 1, wherein said contacting step is carried out at 20-25 degrees Centrigrade.

15. The method of claim 1, wherein said contacting step is carried out below room temperature.

16. The method of claim 1, wherein said contacting step is carried out at 4 degrees Centrigrade.

17. The method of claim 1, wherein said solution further comprises a salt.

18. The method of claim 17, wherein said salt is a sulfate salt.

19. The method of claim 1, wherein said solution further comprises a surfactant.

20. The method of claim 19, wherein said surfactant is non-ionic.

21. The method of claim 1, further comprising homogenizing said albumen fraction

22. The method of claim 21, wherein said homogenizing is carried out at a shear rate in the range of approximately 20 s⁻¹ to approximately 7×10⁶ s⁻¹

23. The method of claim 1, wherein said supernatant is filtered.

24. The method of claim 1, wherein said supernatant is clarified using tangential flow filtration.

25. The method of claim 1, further comprising contacting said supernatant with an ion exchange chromatographic resin.

26. The method of claim 25, wherein said resin is an anion exchange resin.

27. The method of claim 25, wherein said resin is a cation exchange resin.

28. A transgenic non-avian polypeptide composition, wherein said composition is isolated from an avian egg and wherein said composition comprises less than 50% of an amount of total avian polypeptides in an unprocessed egg albumen.

29. The composition of claim 28, wherein said composition comprises less than 50% of the amount of conalbumin in unprocessed egg albumen.

30. The composition of claim 28, wherein said composition comprises less than 50% of the amount of ovalbumin in unprocessed egg albumen.

31. The composition of claim 28, wherein said composition comprises less than 20 g/L of ovalbumin.

32. The composition of claim 28, wherein said composition comprises less than 5 g/L of conalbumin.

33. The composition of claim 28, wherein the ratio of said non-avian polypeptide to total avian proteins is at least 2-fold greater than said ratio in an unprocessed transgenic egg albumen.

34. The composition of claim 28, wherein said polypeptide is a non-avian insulin or insulin precursor polypeptide.

35. The composition of claim 28, wherein said polypeptide is a human insulin or insulin precursor polypeptide.
